(19) United States
(12) Patent Application Publication
(10) Pub. No.: US 2021/0370052 A1
(43) Pub. Date: Dec. 2, 2021

(54) DENTAL IMPLANT AND SYSTEM FOR TREATMENT

(71) Applicant: Garwood Medical Devices, LLC, Buffalo, NY (US)

(72) Inventors: Wayne D. Bacon, Buffalo, NY (US); Brian R. Peterson, East Aurora, NY (US); Timothy M. Kopera, Lancaster, NY (US); Jackson G. Hobble, Buffalo, NY (US)

(21) Appl. No.: 16/884,664

(22) Filed: May 27, 2020

(51) Int. Cl. A61N 1/05; A61C 8/00; A61N 1/20

(52) U.S. Cl. CPC A61N 1/0548; A61N 1/205; A61C 8/0012; A61C 8/0066

(57) ABSTRACT

A dental implant includes a post and an abutment extending from the post, the post and abutment being made from metal. A crown having a hollow cavity is attached onto the abutment, the crown being configured with a metallic insert that can be accessed and coupled to the abutment to enable electrical contact. In one version, the crown includes a metallic core and in another version, the crown can include a formed recess that receives a portion of the metal abutment. In each version, an exposed metallic area is provided, enabling electrical conduction to the abutment and post as part of a bacterial treatment of the dental implant.

### TECHNICAL FIELD

This application is directed to an improved dental implant and related method for treating infected dental implants. More specifically and according to the present invention, treatment can be performed, but without having to first remove the crown portion of the dental implant.

### BACKGROUND

Surgical medical implants are used in patients with many different injuries or medical problems. For example, metal implants may be used for any individual that needs to replace a tooth in a dental procedure.

More specifically and when a patient has a tooth that needs to be removed, the standard procedure is to replace that tooth with a prosthetic by means of a tooth-root replacement. This replacement typically is composed of three (3) main components including a post, an abutment, and a ceramic prosthetic tooth referred to as the crown. The tooth and roots are extracted from the jaw bone of the patient and the bone is reamed to properly fit the post, which is typically metallic. The dental implant is surgically placed in the jaw bone to which it fuses in a process referred to as “osseo-integration”. A healing abutment is placed until the site is sufficiently healed. The healing abutment is then removed, and another metal abutment is screwed onto the post. The prosthetic crown can then be adjoined to the metal abutment. The crown typically is made from a non-conductive material having a hollowed core that allows the abutment to be press fit inside. It is also common to have an abutment that screws into the crown itself. The screw hole is typically at the top of the tooth and then is filled once the complete implant is in place. When completed, the only visible portion in the mouth is the crown.

One potential problem with any surgical metal implant is that they tend to allow for the growth of bacteria on the surface. This may increase the patient's risk for an infection. This issue is especially prevalent in the mouth due to a large bacterial presence. It has been estimated that roughly 20 percent of all subjects receiving a dental implant can develop infections. As bacteria colonize upon foreign surfaces such as metal, they form biofilms. Biofilms are protective extracellular matrix materials that encapsulate bacterial colonies onto a surface and protect them. Biofilms can be 500-5000 times more resistant to antibiotics than common planktonic bacteria because the antibiotics cannot penetrate the biofilm. A significant percentage of dental implants acquire periimplantitis, or biofilm infection of the implant that can cause complications which include implant loosening, gum and bone loss. To decrease the risk of infection, electrodes can provide electrical stimulation to disrupt the growth of bacteria. It has been shown in scientific literature that the application of cathodic current to metal samples create chemical reactions at that surface that can disrupt and kill bacterial biofilms formed on the surface of the metal implant.

Electrical connection to the dental implant to conduct a treatment can be difficult due to the non-conductive crown that sits on top of the metal post and abutment, with the crown being located above the gum line. Alternative and less desirable approaches to electrically connect to the metallic post include removing the crown or using a needle to pierce through the gum. Each alternative has several issues that make it largely impractical. Accordingly, there is a need in the field to enable a biofilm treatment procedure to be conducted upon an infected metal dental implant, but without having to first remove the crown in advance of treatment.

### BRIEF DESCRIPTION

The present invention relates to the use of voltage-controlled electrical treatment to the surfaces of a metal implant, such as a dental implant, as a method to prevent and eradicate microbial colonization on the surface, such as periimplantitis. This invention is implemented by application of a DC electrical current to a metallic implant. The system requires at least two electrodes, but can also utilize three or more electrodes. Specifically and in the case of three electrodes, the system includes a counter electrode, a working electrode, and a reference electrode. The counter electrode delivers the current to the working electrode to maintain a steady DC potential with respect to the stable reference electrode. In the case of a dental implant, the metallic surface of the implant post and abutment act as the working electrode. The system uses the electrochemical properties of the at least two electrodes in a direct current (DC) circuit to disrupt and chemically kill the biofilm, which means the electrodes must be submerged or contacting an electrolyte that transports the electrical energy through chemical reactions to the other electrode(s). Human bone and soft tissue provide this electrolyte media for conduction, and thus the complete surface area of the dental implant embedded in the bone receives treatment. Full surface treatment optimizes effectiveness against biofilm infections.

The present disclosure provides a means for making an electrical connection to a metallic post and abutment of a specialized dental implant. The crown-abutment assembly of the implant improves the ease and efficiency of treatment using cathodic voltages. When applying therapy to an infected dental implant, it is preferable to keep the crown attached as opposed to alternative attachment mechanisms that require direct attachment to the abutment and removal of the crown. The herein described implant contains an easily accessible conductive contact point that provides full electrical conduction to the remainder of the implant while the crown remains attached.

Therefore and in accordance with at least one aspect, a novel apparatus is described that enables reliable electrical connection to the metal abutment and post of a dental implant in order for the chemical reaction to proceed safely and effectively. According to one version, a metal core is incorporated within the crown and coupled to the abutment, wherein the core is configured to create an exposed metal contact point or area on the surface of the crown that provides a simple but effective way to electrically connect to the metal post and abutment of the dental implant. Treatment of the dental implant is enabled, but without resorting to removal of the crown in order to perform the treatment.

In one version, the crown includes the metallic core in which a portion of the core extends transversely relative to a center axis of the post to a side wall of the crown. The transverse portion extends through an opening formed in the crown as the exposed metallic area, which is configured for receiving a needle or other electrically conductive member coupled to a potentiostat or other similar device capable of delivering an electrical potential. Aesthetically, the exposed metallic area can be provided on an inner side wall of the crown. Alternatively, the exposed area of the metallic core can extend upwardly through a top surface of the dental crown.

According to another version, the hollow crown can be matched to the abutment of the dental implant using a key-shaped or similar form of configuration. More specifically, a key-shaped extension of the abutment is shaped and configured for disposition within a recess formed in a side wall of the dental crown, thereby providing an exposed and accessible metallic area. It will be understood that other suitable versions and configurations can be made in accordance with various aspects of the invention.

According to another aspect of the present invention, a method is provided for enabling treatment of a dental implant in order to disrupt bacterial deposits using cathodic voltages, the dental implant comprising a metallic post secured to the jaw bone of a subject and a prosthetic tooth or crown, made from a non-conductive material attached onto a metal abutment of the metallic post, the method comprising:
a. configuring the crown of the dental implant with a metal insert such that a portion of the metal insert extends as an exposed surface area or zone along a wall of the crown;
b. creating electrical contact between the metal insert and the metal abutment of the dental implant;
c. electrically contacting the exposed surface area or zone of the crown to a source capable of producing an electrical potential in which the implant becomes a working electrode; and
d. electrically contacting at least one other electrode relative to the implant and the source capable of producing an electrical potential, thereby creating an electrochemical reaction.

An advantage is that the herein described implant and related method are very minimally invasive for purposes of treating infections. Accordingly, the herein described techniques are less time consuming, while yet still being reliable.

In addition and as described herein, the dental implant can be used in conjunction with various treatment systems used for disrupting biofilms, the systems including 2, 3, 4 or more electrodes and with the dental implant being used as a working electrode without removal of the crown.

These and other features and advantages will be readily apparent from the following Detailed Description, which should be read in conjunction with the accompanying drawings.

### BRIEF DESCRIPTION OF THE DRAWINGS

FIG. 1(a) is a side elevational view of a dental implant made in accordance with a first embodiment according to aspects of the present invention;

FIG. 1(b) is a sectioned elevational view of the dental implant of FIG. 1(a) taken through Section A-A;

FIG. 1(c) is a partial perspective exploded view of the dental implant of FIGS. 1(a) and 1(b), including the dental crown and abutment portions;

FIG. 2(a) is a side elevational view of a dental implant made in accordance with a second embodiment according to aspects of the present invention;

FIG. 2(b) is a partial perspective exploded view depicting the dental crown and abutment portions of the dental implant of FIG. 2(a);

FIG. 3(a) is a block diagram of a treatment system made in accordance with an embodiment;

FIG. 3 (b) is a block diagram of a treatment system made in accordance with another embodiment; and

FIG. 3(c) is a block diagram of a treatment system made in accordance with yet another exemplary embodiment.

### DETAILED DESCRIPTION

The following relates to certain exemplary embodiments of a dental implant configured for electrical treatment in the case of infection using cathodic voltages as well as treatment systems used with the implant. It will be understood that various modifications are possible. In addition, various terms are used throughout the following discussion in order to provide a suitable frame of reference for the accompanying drawings. These terms, which may include “first”, “second”, “third”, “upper”, “lower”, “internal”, “external”, “inner”, “outer” and the like, are not intended to be overly limiting of the present invention, except where so specifically indicated.

The accompanying drawings are described throughout in order to convey salient features of the present invention, but the drawings should not be relied upon for scaling or similar purposes.

The herein described system and method relies upon the introduction of an electrical current to an electrochemical cell. As a matter of background and for electrochemical(redox) processes to occur, there must be an anode and a cathode within an electrolyte solution. The anode is a metallic surface where oxidative reactions occur, and the cathode is another metallic surface where reduction reactions occur. A reduction reaction occurs when the material of interest gains electrons and thereby decreases the oxidation state of the molecules. The electrolyte that the electrodes each reside in provides the electrical connection by facilitating the flow of electrons shuttled by ion carriers, such as electrolytic sodium or potassium ions. Electrons are driven from the anode to the cathode through the electrical path via a potentiostat or similar device. More specifically, a potentiostat is an instrument used to drive current from a counter electrode to a working electrode in order to keep the voltage on the working electrode at a constant value compared to a stable reference electrode. One such procedure used for the treatment of biofilms on a metallic implant is described in U.S. Pat. No. 9,616,142, the entire contents of which are herein incorporated by reference.

According to this treatment procedure, the anode represents the counter electrode and the cathode represents the working electrode. Using a potentiostat, a user can dictate which electrochemical process is occurring on the working electrode and at what rate the process occurs simply by adjusting the applied voltage parameters with respect to a separate reference electrode. The cathodic reactions occurring at the working electrode produce hydroxide ions, resulting in an alkaline pH at on the implant surface, while also producing different reactive oxidative chemical species that are bactericidal for existing biofilms.

In a clinical setting, the above-noted technique has been shown as a way to fight bacterial biofilm infections on metallic implants in the most minimally invasive way possible. In this setting, the patient's body can act as an electrochemical cell by using the metal implant as the cathode and the counter electrode as the anode. It has been shown that the above techniques can be used for the treatment of various orthopedic implants as well as dental implants. As noted above, an issue is the dental crown, the latter being made from a non-conductive material covering the surgically implanted metallic post.

Removing the dental crown is an option that many dentists prefer not to perform because the crown may break or cause extra trauma to the afflicted tissue. In the case where a biofilm exists on the post and abutment, the flow of electrons into the bulk metal, out the metal surface, and into the electrolytic environment, will create bactericidal chemical species that attack the biofilm from the metal surface outwards. pH is also a large factor in the bactericidal effect as laboratory testing has shown that microenvironment pH levels microns away from the surface can become alkaline, as high as a pH of 12, within minutes of electrical stimulation.

In brief and according to this application, a dental crown is provided that increases the efficiency of the above-noted form of treatment as it pertains to a dental implant. The improved and novel dental crown allows the dentist to access the fully bone-embedded implant surface for use as the working electrode for electrical stimulation, while still maintaining patient safety parameters and a minimally invasive profile.

The following embodiments are exemplary design versions of a dental crown that has been configured with an exposed metal contact surface on the side or other surface of the crown that is electrically continuous through the abutment and to the embedded post of a dental implant.

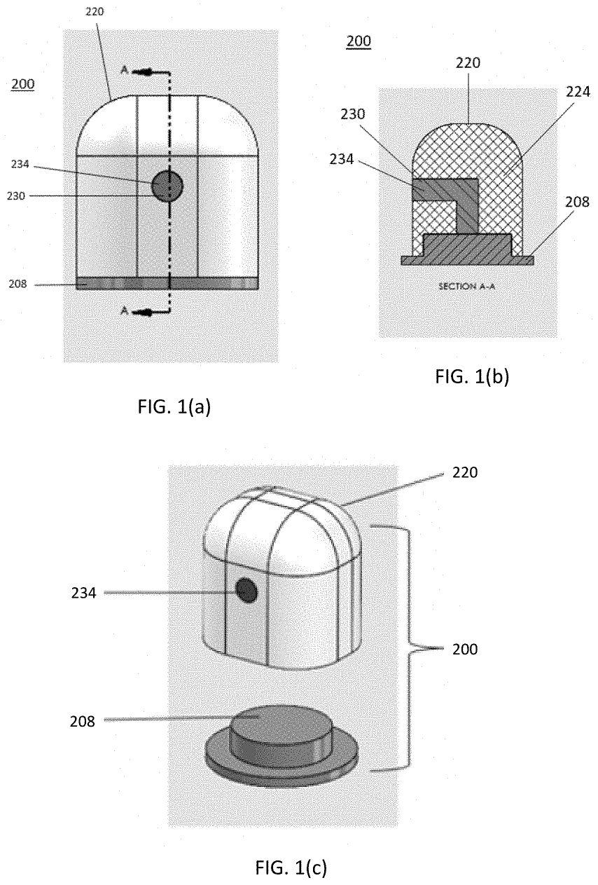

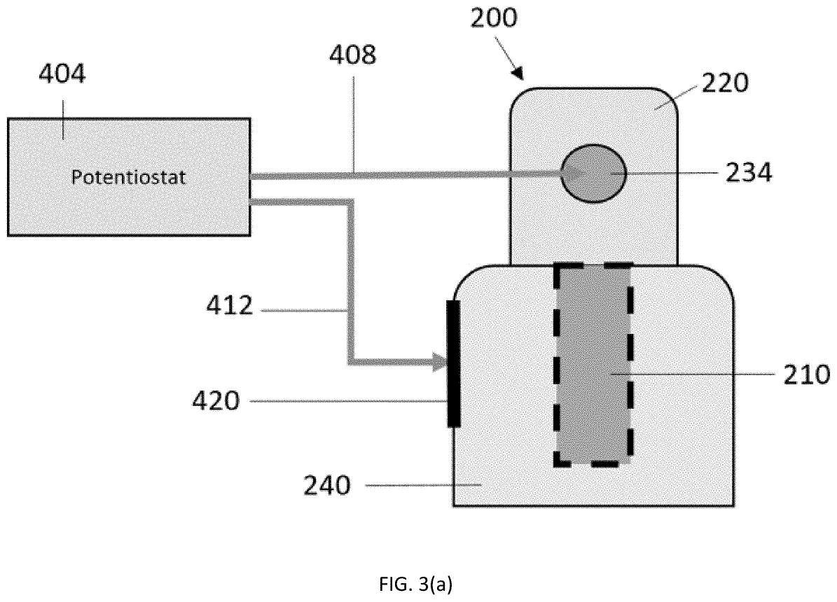

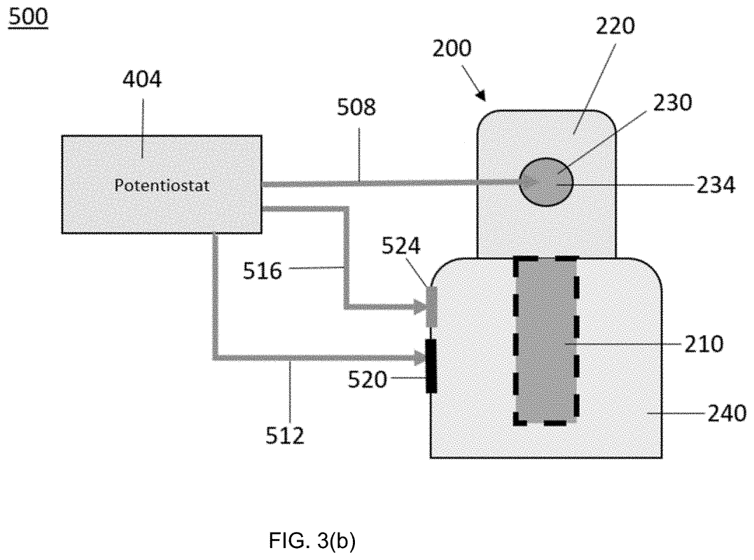

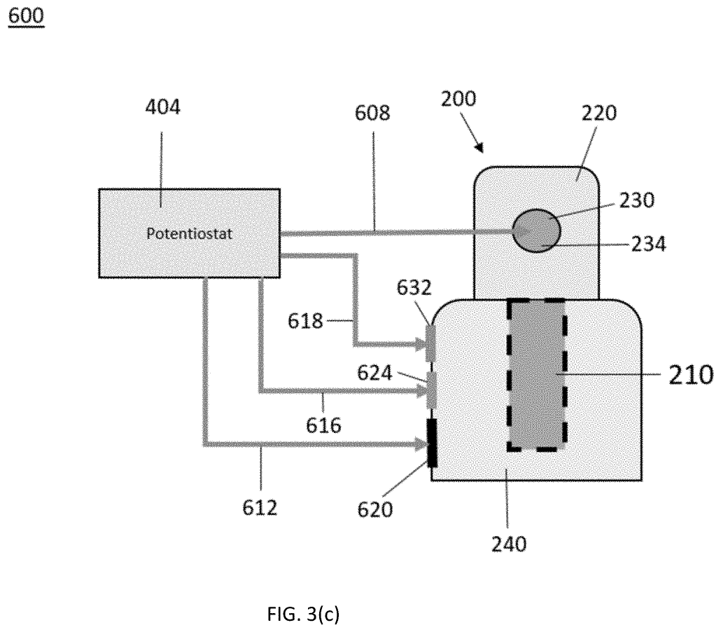

Referring to FIGS. 1(a)-1(c), the dental implant 200 according to this embodiment includes a metal abutment 208 and a dental crown 220, the latter being made from a non-conductive material such as ceramic, porcelain or a suitable polymer having an open end and an enclosed hollow cavity 224 that is suitably sized and shaped to be fitted over the metal abutment 208. The abutment 208 is tied to a metallic post 210, the latter component being shown more clearly in FIGS. 3(a)-3(c), wherein the metallic post 210 is fused directly to the jawbone 240 of the patient, also shown schematically in FIGS. 3(a)-3(c). According to this embodiment, the metal abutment 208 is press fit into the crown 220, the latter having an integrated metallic core 230. The metallic core 230 is made from a suitable metallic material that enables and facilitates electrical conduction. In a preferred embodiment, the metallic core 230 of the crown 220 is composed of a biocompatible metal commonly used in dentistry, such as but not limited to titanium, stainless steel and/or their alloys. When the metal abutment 208 is surgically implanted, the abutment 208 is placed into direct physical contact with a distal end of the metallic core 230, thereby creating an electrical connection. The metallic core 230 can alternatively be made integral with the abutment 208 (or can be formed as part of the crown 230), according to this embodiment.

More specifically and according to this specific embodiment, the metallic core 230 extends upwardly as shown more specifically in FIG. 1(b) through the hollow cavity 224 of the dental crown 220, including a transverse portion of the core 230 that further extends to an opening formed in a side wall of the dental crown 220. A proximal end of the metallic core 230 is preferably flush with the side wall of the dental crown 220, thereby exposing a small metallic surface zone 234 on the dental crown 220, as shown. The shape of the exposed metallic surface zone 234 is circular according to this specific embodiment, but it will be understood that the shape of the defined zone 234 and the metallic core 230 can be suitably varied. Making the proximal end of the core 230 flush to the surface of the crown 220 is preferable so not to cause any overhang or sharp edges. According to one version, the exposed surface area 234 of the metallic core 230 may be dimpled to allow for better mating with the crown attachment mechanism. In a preferred embodiment, the exposed metallic surface zone 234 resides on the inner facing wall of the crown 220 relative to the patient, such that the exposed metallic surface zone 234 is not visible.

The overall shape and configuration of the metallic core 230 can be suitably varied provided that the dental crown 220 can adequately and structurally function primarily as a prosthetic tooth. For example and in lieu of extending transversely as shown, the proximal end of the metallic core 230 can extend vertically through the hollow cavity 224 until exposed at a top surface of the crown 220.

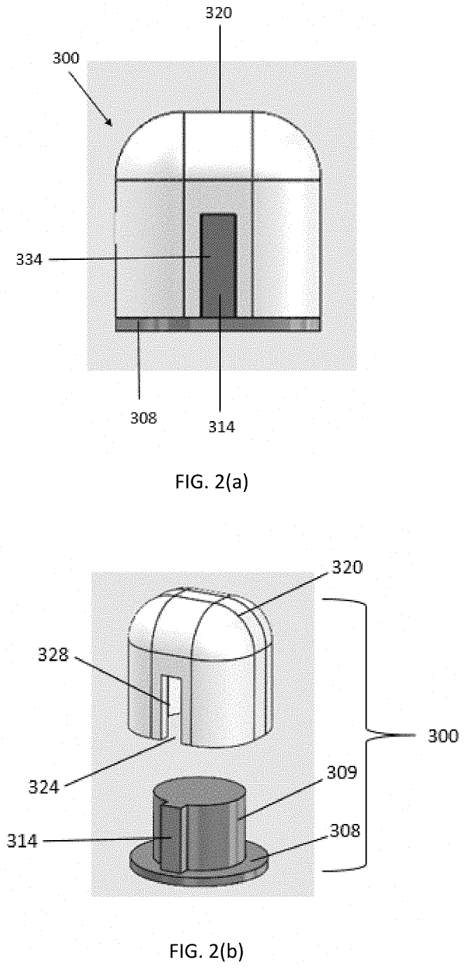

FIGS. 2(a) and 2(b) depict various views of a novel dental implant 300 made in accordance with another exemplary embodiment. Similar parts are herein labeled with the same reference numbers for the sake of clarity. As in the preceding, the dental implant 300 includes a metallic post 210, FIG. 3(a), that is fused to the jawbone 240, FIG. 3(a) of the patient with a metal abutment 308 being disposed above the post 210. Each of the abutment 308 and the post 310 are made from a suitable metal, such as stainless steel, titanium or an alloy thereof. A dental crown 320, made from a nonconductive material includes a hollow cavity 324 that is sized to be fitted onto the metal abutment 308. In this particular version, the metal abutment 308 is defined by a central body 309 having a substantially circular shape including a radially extending extrusion 314. More specifically, the metal abutment 308 is shaped and configured to fit into the hollow cavity 324 of the dental crown 320 with the radially extending extrusion 314 being key-shaped and configured to be fitted within a matching recess 328 formed in a side wall of the dental crown 320 for purposes of engagement. Preferably, the recess 328 is formed on an inner facing side of the dental crown 320.

In this embodiment and unlike the version shown in FIGS. 1(a)-1(c), the dental crown 320 does not have an integrated metallic core and includes only the formed recess 328 that is sized to receive the key shaped extrusion 314 of the metal abutment 308. The length of the key shaped extrusion 314 extending radially outward from the central body 309 of the abutment 308 is preferably of a specific length to insure that the extrusion 314 is flush with the surface of the side wall of the crown 320 to avoid overhangs or sharp edges.

Like the previous embodiment, this implant design introduces an exposed metal surface or zone 334 on the side of the dental crown 320 that allows direct electrical connection to the abutment 308 and post 210, FIG. 3(a), without first requiring removal of the crown 320 from the remainder of the dental implant 300. This specific embodiment may be advantageous, for example, if the dentist chooses to use any cement within the inner cavity 324 of the dental crown 320.

In each of the herein described embodiments, the exposed metallic contact zone 234, 334 is introduced on a surface of the crown 220, 320 that provides the dentist access in order to apply a cathodic voltage as part of a treatment that can effectively disrupt and eliminate biofilm from a dental implant, but without first removing the crown 220, 320.

As shown in FIGS. 3(a)-3(c), the herein described dental implants can be used in conjunction with various cathodic voltage-based treatment configurations or systems, herein labeled 400, 500, and 600, respectively. For purposes of this discussion, the dental implant 200 is shown in use with each configuration 400, 500, and 600, shown schematically for illustrative purposes. Though not described in detail, it will be understood that the dental implant 300 can be similarly used in conjunction with any of these treatment systems.

Each cathodic voltage treatment system 400, 500 and 600, shown diagrammatically in FIGS. 3(a)-3(c), respectively, commonly includes a potentiostat 404, the latter being capable of generating an electrical potential as well as a number of electrodes that minimally include a working electrode and a counter electrode. The minimal configuration is shown with reference to FIG. 3(a) and in the first treatment system 400, a pair of electrodes are employed that include a working electrode and a counter electrode 420. The working electrode is the dental implant 200 based on the availability of the exposed metallic surface area 234 of the crown 230, while the counter electrode 420 is preferably made up of silver/silver chloride although other materials can be used. The counter electrode 420 is attached to the gum/jawbone 240 area of the patient via an electrical lead or wire 412 coupled to the potentiostat 404. An electrical lead 408 is further provided extending to a needle or other conductive member that is placed into contact with the exposed metallic area 234 of the crown 230. Electrochemical current is caused to flow based on voltages applied by the potentiostat 404 via an electrochemical circuit being formed between the working electrode 200 and the counter electrode 420. Due to the exposure area 234, the potentiostat 404 is electrically connected to the metal abutment 208, and thus the entire post-abutment-core system is electrically connected. However, the only metallic materials that are in contact with a conductive electrolyte (bone and soft tissue) are the metal abutment 208 and the post 210. Because the metallic core 230 is encapsulated by the non-conductive crown material, the metal core 230 essentially acts as an electrical wire or lead capable of transferring electrical energy (i.e., current) from the potentiostat lead to the dental implant 200. This system 400 provides a significant advantage, when compared to other comparable potentiometric treatment systems or techniques, because the dental crown 220 does not have to be first removed in order to perform treatment.

FIG. 3(b) diagrammatically illustrates a three (3) electrode system 500 that includes the working electrode (the implant 200) as well as a counter electrode 520 functioning in the same manner as that of the prior system 400 and coupled to the potentiostat 404 via electrical leads 508 and 512, respectively. A third (reference) electrode 524 is applied along with the counter electrode to the gums/jawbone area 240 of the subject and in electrically coupled to the potentiostat via lead 516. The reference electrode 524 permits greater electrochemical control for the treatment system 500. In a preferred embodiment, the reference electrode 524 is made from silver/silver chloride, thus producing a stable electrochemical biopotential for the working electrode (implant 200). Further details relating to the potentiostat and counter and reference electrodes are described in greater detail in U.S. Pat. No. 9,616,142, previously incorporated by reference in its entirety.

FIG. 3(c) diagrammatically illustrates yet another version of a treatment configuration 600 that employs four (4) electrodes. As in the preceding, the dental implant 200 due to the exposed metallic area 234 acts a working electrode as electrically coupled to the potentiostat 404 by electrical lead 608. A counter electrode 620 and reference electrode 624 are attached to the gum/jawbone area 240 of the patient as connected to the potentiostat 404 by leads 612 and 616, respectively. Each of the electrodes 620 and 624 operate in the same manner as those previously described. In addition, this treatment system 600 is further equipped with a working sense electrode 632, similarly attached to the gum/jawbone 240 area of the patient and coupled to the potentiostat 404 by electrical lead 618. The working sense electrode 632 enables further control and data feedback of the working electrode (implant 200). Other suitable cathodic voltage treatment configurations or systems can also be utilized. In addition and though the counter, reference and working sense electrodes are shown according to this embodiment being attached to the jawbone/gum area 240, FIGS. 3(a)-3(c) of the patient, other suitable positioning of these electrodes is permitted. For example, any or all of these electrodes could also be located outside the mouth on the face or completely external to the body and connected via a salt bridge.

Each of the above systems/configurations permit reliable treatment of the dental implant, but without requiring removal of the crown. The exposed metallic surface area of the crown permits electrical conduction to the remainder of the dental implant. Exposed metal surfaces are both safe and cosmetically acceptable when applying these designs and embodiments. A distinct differentiator from alternative dental treatment techniques is that the herein described implant promotes conduction over the entire bone-embedded surface of the dental implant, and not just conduction, for example, within the abscess pocket. This differentiator is a significant advance, especially in regard to dental implant posts. Implant posts are typically manufactured with a very rough, coarse microsurface to promote osseointegration. One issue this microsurface can create is that bacteria are able to “hide” within the crevices of the microstructure, even when bone matrix are apparently grown into the surface. The approach and design of herein described apparatus allows for thorough treatment of all microstructures in the metal, even with bone present, to eliminate all bacteria from those locations. It has been found and substantiated in scientific literature that at optimized treatment parameters, matrix embedded bone cells that are local to the reaction are not affected to a high degree.

Other alternative techniques for treatment of conventional dental implants are available but each of the techniques are believed to be inferior to the implants described according to the present invention. A pair of these alternatives are briefly described as follows, each alternative including two electrodes that can be used to deliver a cathodic voltage treatment in which the metal implanted post and abutment represent the working electrode along with a counter electrode.

In the first alternative, a needle is used to pierce through the gum to make physical contact with the post, creating a working electrode connection. A small tube is the inserted into the abscess of the infection to pump electrolyte into the abscess. The electrolyte from the tube acts as a conductive bridge to an external electrode provided in the tube or at the other terminal of the tube. The disadvantages of this setup include executing a secure attachment to a small embedded implant with a needle, and the effectiveness of the treatment based on the electrode set up. Needle placement in this fashion will be difficult and could easily disconnect during treatment. Also, with the electrolyte from the tube acting as the main transport path, the treatment will be locally concentrated only in the open abscess and not on the whole bone to metal interface where additional biofilm may exist.

The second alternative treatment technique requires removal of the dental crown to electrically connect the electrodes to the infected metal post. Once the crown is removed, a small electrolyte-filled chamber is attached to the implant that flows electrolyte downward onto the implant and abscess. The counter electrode exists inside the chamber and therefore the formed conductive path is from the dental implant back up the electrolyte and into the chamber. Each of the above-noted alternatives commonly require an added electrolyte to connect to the external counter electrode. The disclosed invention demonstrates advantages over these alternatives due to a minimally invasive and secure profile that doesn't require removal of the crown. The disclosed invention also improves the efficacy of the therapy by using the local bone and body tissue as the conductive path for a more evenly distributed and thorough treatment.

### PARTS LIST FOR FIGS. 1(a)-3(c)

200 dental implant
208 abutment
210 post
220 crown
224 hollow cavity, crown
230 metallic core
234 metallic surface area or zone
240 jawbone
300 dental implant
308 abutment, metal
309 central body, abutment
314 radially extending protrusion, abutment
320 dental crown
324 hollow cavity, crown
328 recess
334 metallic surface area or zone
400 treatment system or configuration
404 potentiostat
408 lead, electrical
412 lead, electrical
420 counter electrode
500 treatment system or configuration
508 lead, electrical
512 lead, electrical
516 lead, electrical
520 counter electrode
524 reference electrode
600 treatment system or configuration
608 lead, electrical
612 lead, electrical
616 lead, electrical
618 lead, electrical
620 counter electrode
624 reference electrode
632 sense electrode

It will be understood that modifications and variations of the described embodiments are possible and within the intended scope of the invention and in accordance with the following claims.

1. A dental implant comprising:
a post;
an abutment extending from the post, the post and abutment each being made from metal in which the post is surgically implanted within the jaw bone of a patient; and
a crown having a hollow cavity disposed onto the abutment, and in which the crown is configured with an exposed metallic surface area that enables electrical conduction to the abutment and post.

2. The dental implant according to claim 1, in which the crown comprises a metallic core within the hollow cavity, the metallic core having a distal end in direct contact with the abutment and an opposing proximal end that extends through a wall of the crown.

3. The dental implant according to claim 2, in which the proximal end of the metallic core is substantially flush to the wall of the crown.

4. The dental implant according to claim 3, in which the wall is a side wall of the crown.

5. The dental implant according to claim 4, in which the wall is an inner side wall of the crown.

6. The dental implant according to claim 3, in which the wall is the top surface of the crown.

7. The dental implant according to claim 2, in which the crown comprises a recess formed in a side wall, the abutment further comprising a central body having a protruding key-shaped portion that is shaped to fit within the crown recess.

8. A method for enabling treatment of a dental implant in order to disrupt bacterial deposits using cathodic voltages, the metallic post comprising a post secured to the jaw of a subject and a prosthetic tooth or crown attached onto a metal abutment of the metallic post, the method comprising:
a. configuring the crown of the dental implant with a metal insert such that a portion of the metal insert extends as an exposed surface area or zone along a wall of the crown;
b. creating electrical contact between the metal insert and the metal abutment of the dental implant;
c. electrically contacting the exposed surface area or zone of the crown to a source capable of producing an electrical potential in which the implant becomes a working electrode; and
d. electrically contacting at least one other electrode relative to the implant and the source capable of producing an electrical potential, thereby creating an electrochemical reaction.

9. The method according to claim 8, wherein the metal insert is provided as a portion of the metal abutment that extends into a formed recess of a side wall of the crown, forming the exposed surface area or zone.

10. The method according to claim 8, in which the metal insert is disposed within a hollow cavity of the crown, the metal insert including a portion that extends transversely to a major axis of the metal post and through an opening formed in the side wall of the crown.

11. The method according to claim 9, in which the abutment includes a central body and a radially extending protrusion, the radially extending protrusion being a key sized and shaped to extend into the recess of the crown, the latter acting as a keyway.

12. The method according to claim 8, wherein one end of the metal insert extends through the crown through an opening at a top surface of the crown, the one end being substantially flush to the top surface.

13. The method according to claim 8, including a counter electrode and a reference electrode, each being electrically connected to the working electrode.

14. The method according to claim 8, further comprising a counter electrode, a reference electrode and a working sense electrode, each electrically connected to the working electrode.

15. A crown for a dental implant comprising:
a hollow body configured to be disposed over an abutment of a metallic post implanted within the mouth of a subject, the crown being configured to enable electrical contact with the abutment to enable treatment of the dental implant without removal of the crown from the subject.

16. The crown according to claim 15, further comprising:
a metallic core disposed within the hollow body, the metallic core having one end configured to be in contact with the abutment and an opposing end accessible as an exposed metallic area through one wall of the body to enable application of an electrical potential.

17. The crown according to claim 16, in which the exposed metallic area is substantially flush with a side wall of the body.

18. The crown according to claim 16, in which the exposed metallic area of the core extends through a top surface of the body.

19. The crown according to claim 16, in which the metallic core includes a vertical section having the end configured for contact with the abutment and a horizontal section transversely arranged relative to the vertical section including the exposed metallic area.

20. The crown according to claim 15, in which the crown includes a recess formed in a sidewall of the hollow body that is configured to receive a portion of the abutment and enable direct access to the abutment without requiring removal of the crown for treatment.
